(19) United States
(12) Patent Application Publication
(10) Pub. No.: US 2004/0161520 A1
(43) Pub. Date: Aug. 19, 2004

(54) Process for removing oil and/or sugar from lecithin

(76) Inventor: Jonathan Maynes, Fort Wayne, IN (US)

Correspondence Address: Richard B. Taylor, James L. Cordek, Solae, LLC, P.O. Box 88940, St. Louis, MO, 63188, US

(21) Appl. No.: 10/625,820

(22) Filed: Jul. 23, 2003

Related U.S. Application Data
(60) Provisional application No. 60/397,822, filed on Jul. 23, 2002.

(51) Int. Cl. A61K 31/685; C07F 9/02; A23J 7/00

(52) U.S. Cl. 426/601; 554/80; 514/78

(57) ABSTRACT

An improved lecithin product is provided by a process for extracting oil and/or sugar from crude lecithin. The improved lecithin product has up to about 75 wt. %, phospholipids, and an oil and sugar content of about 10 wt. % or less. The improved lecithin product may have acetone insolubles content of more than about 68, and sugar content of less than about 1.0 wt. %. The process provided involves extracting oil and/or sugar from crude lecithin using blends of ethanol and water in two extraction steps. The method includes: providing a crude lecithin material; mixing the crude lecithin material with a blend of ethanol and water in a first extraction step; collecting the residue from the first extraction step; mixing the residue from the first extraction step with a blend of ethanol and water in a second extraction step; and collecting the residue from the second extraction step as an improved lecithin product.

### CROSS REFERENCE TO RELATED APPLICATIONS

This application claims the benefit under Title 35, U.S.C. § 119(e) of U.S. Provisional Patent Application Serial No. 60/397,822, entitled PROCESS FOR LECITHIN DE-OILING, filed on Jul. 23, 2002.

### BACKGROUND OF THE INVENTION

1. Field of the Invention

This invention relates to the art of extracting oil and/or sugar from soybean material while retaining phospholipids in the soybean material. More particularly, this invention relates to ethanol and water extraction of oil and/or sugar from crude lecithin while retaining phospholipids in the lecithin.

2. Description of the Related Art

The benefits of soy protein and lecithin are well documented. Cholesterol is a major concern for consumers throughout the industrialized world. It is well known that vegetable products contain no cholesterol. For decades, nutritional studies have indicated that the inclusion of soy protein and lecithin in the diet actually reduces serum cholesterol levels in people who are at risk. The higher the cholesterol, the more effective soy proteins are in lowering that level. Additional health benefits of lecithin have also been well demonstrated.

Soybean is known to have the highest protein content of all cereals and legumes. In particular, soybean has about 40 wt. % protein, while other legumes have 20-30 wt. %, and cereals have about 8-15 wt. % protein. Soybean also contains about 20 wt. % oil with the remaining dry matter mostly carbohydrate (35 wt. %). On a wet basis (as is), soybean contains about 35 wt. % protein, 17 wt. % oil, 31 wt. % carbohydrates including sugars, and 4.4 wt. % ash.

In soybean, both storage protein and lipid bodies are contained in the usable meat of the soybean (called the cotyledon). The complex carbohydrate (or dietary fiber) and phopholipids are also contained in the cell walls of the cotyledon. The outer layer of cells (called the seed coat) makes up about 8 wt. % of the soybean's total weight. The raw, dehulled soybean is, depending on the variety, approximately 18 wt. % oil, 2 wt. % lecithin, 15 wt. % soluble carbohydrates including sugars, 15 wt. % insoluble carbohydrates, 14 wt. % moisture and ash, and 38 wt. % protein.

In processing, soybeans are carefully selected for color and size. The soybeans are then cleaned, conditioned (to make removal of the hull easier) and cracked, dehulled and rolled into flakes. The flakes are subjected to a solvent bath that removes the oil. The solvent is removed and the flakes are dried, creating the defatted soy flakes that are the basis of all soy protein products.

The oil laden solvent that is removed is then further processed to refine oil. In the oil refining process, lecithin is extracted and processed for further commercial value.

The mixture of phosphatides referred to in the trade as lecithin is a group of phospholipids composed of the following structural members: glycerol, fatty acids, phosphoric acid, amino alcohols and carbohydrates. These phospholipids are found in practically any animal and vegetable material. Ample amounts thereof are present in brain tissue, egg yolk, and oil seeds such as soybean oil and rape-seed oil.

As a result, lecithin can be obtained from a variety of sources, for example eggs, brain tissue, plant seeds and beans, e.g. soybeans and sunflower seeds. Lecithin normally comprises a variety of phosphatides, in particular phosphatidyl choline (PC), phosphatidyl ethanolamine (PE), phosphatidyl inositol (PI) and phosphatidic acid (PA). For example, commercial soybean lecithin commonly comprises from about 45% to about 55% by weight of phosphatides. These phosphatides consist mainly of PC, PE, PI and PA, each of which is commonly present in amounts of from about 8 wt. % to about 20 wt. % of the lecithin. Smaller amounts of other phosphatides, e.g. phosphatidyl serine, are present from about 30 to about 40 wt. % of triglycerides and other lipids. Smaller amounts of other components, e.g. sugars are also present.

Medical evidence that phosphatidyl choline may be of value in treating certain health problems and the growing popularity of health foods has increased the demand for palatable foods that contain a high level of phosphatidyl choline, and the demand for lecithin as a supplement.

It has been determined that lecithin optimizes physiological functions and restores impaired physiological functions in situations associated with inadequate cholinergic transmission such as tardive dyskinesia, manic depressive states or other psychiatric diseases, memory impairment, familial ataxias or the like.

The positive effects of lecithin in reducing arterial plaque, decreasing LDL cholesterol, and increasing HDL cholesterol, are well known and documented.

It is known to separate oil from crude lecithin by extraction processes that use acetone. Lecithin obtained through acetone extraction must be dried at temperatures below 70° C., otherwise thermal decomposition of the phospholipids will occur. Even at low temperatures, there will be formed in the course of the drying operation acetone-induced products which impact the organoleptic quality of the reduced oil and sugar phospholipids and cause a musty hay-like odor and a sharp pungent aftertaste in products produced therefrom.

It is also known to separate oil from crude lecithin by extraction processes that use carbon dioxide and ethane, carbon dioxide and propane, and volatile hydrocarbons. It is also known to separate oil from crude lecithin by making use of membranes to separate the oil and phospholipids based on size characteristics. Each of these processes has its own disadvantages.

### SUMMARY OF THE INVENTION

The present invention provides an improved lecithin product having a substantially reduced level of sugar, or oil and sugar, and having a desirable flavor and functional properties. The improved lecithin product is high in phospholipids without adverse extraction solvent flavor or color impairment. The method for producing the improved lecithin product employs a blend of ethanol and water as the extraction solvent, and avoids the use of extraction solvents that impair the flavor and/or color of lecithin products.

An improved lecithin product is provided having up to 75 wt. % phospholipids, and an oil and sugar content of about 10 wt. % or less.

Another improved lecithin product provided has an acetone insolubles (AI) content of more than about 68 and a sugar content of less than about 1.0 wt. %. “Acetone insolubles” refers to a measurement of the amount of acetone insoluble matter which is an indication of purification. Acetone insolubles can be determined by one skilled in the art using American Oil Chemists Society Method (AOCS) pp 4-46.

A method for producing an improved lecithin product having a high phospholipid content, a low sugar content, and optionally low oil content is provided. The method includes the steps of: providing a crude lecithin material; mixing the crude lecithin material with a blend of ethanol and water to form a first mixture in a first extraction; retaining solids from the first extraction; mixing the retained solids with a blend of ethanol and water to form a second mixture in a second extraction; and retaining solids from the second extraction as an improved lecithin product.

It is an object of this invention to provide a novel lecithin product with a high phospholipid content from soybean conventionally grown by farmers and used by soybean processors. The lecithin product has a high phospholipids content or a high acetone insolubles content.

It is a further object of this invention to provide an improved lecithin product that has an oil and sugar content of about 10 wt. % or less and without the use of extraction solvents that impair the flavor and/or color of the lecithin product.

It is yet a further object of this invention to provide an improved lecithin product having a high phospholipid content and substantially reduced sugar content.

It is another object of this invention to be able to control the manufacturing process to achieve a desired, improved lecithin product with a high phospholipid content without imparting an undesirable flavor and/or color to the lecithin product. In particular, it was discovered that by using a blend of ethanol and water as an extracting solvent, a lecithin product could be produced with a high phospholipid content without imparting an undesirable solvent impaired flavor and/or color to the lecithin.

It is a further object of this invention to produce an improved lecithin product by an economically efficient method.

### DETAILED DESCRIPTION

An improved lecithin product is provided having a phospholipid content of up to about 75 wt. %, preferably about 75 wt. %, and a desirable flavor profile. The improved lecithin product has a phospholipid profile that includes significant amounts of phosphatidyl-choline (PC), phosphatidyl-ethanolamine (PE), and phosphatidyl-inositol (PI). The sugar or oil and sugar content of the improved lecithin product is substantially reduced.

A method for producing an improved lecithin product provided generally includes the steps of: providing crude lecithin; mixing the crude lecithin with a blend of ethanol and water to extract oil and/or sugar; and mixing the residual material a second time with a blend of ethanol and water to extract sugar.

Crude lecithin used as a starting material herein is a complex, naturally occurring mixture of phosphatides that are isolated from vegetable products by degumming the corresponding vegetable oil with, for example, a small quantity of steam or water. The phospholipid composition produced, also known as lecithin sludge, generally contains 8 to 59% by weight phospholipids. The phospholipid composition may be dried by one or more of various methods to yield crude lecithin.

Each initial crude lecithin used in the process of the present invention may have a varying chemical composition, depending on the source of the crude lecithin. The crude lecithin obtained from soybeans, i.e. soy lecithin, after drying, is composed of the following components, by weight percent:

| | | | |
|---|---|---|---|
| Triglycerides | 34.2% | phosphatidyl-choline | 19.1% |
| Dilycerides | 0.4% | lysophosphatidyl-choline | 0.7% |
| Free fatty acids | 0.4% | phosphatidyl-ethanolamine | 8.6% |
| Other neutral lipids | 10.8% | phosphatidyl-inosito | 18.8% |
| Glycolipids | 6.5% | phosphatidic acid | 4.2% |
| Carbohydrate | 6.7% | N-acylphosphatidyl- | 1.0% |
| | | ethanolamine | |
| | | others | 8.6% |

In the first extraction step, the crude lecithin is contacted with a blend of ethanol and water by mixing the crude lecithin into the ethanol/water blend. The ratio of the lecithin to the ethanol/water blend may range from about 1:1 to 1:5. However, a ratio of 1:3 may yield good results. The ratio of ethanol to water in the blend may range from about 1:1 or about 5:4, although other ratios may also be suitable. Mixing is conducted in a shear mixer in order to ensure that the lecithin is broken up sufficiently. The shear mixer may be a high shear mixer or a low shear mixer. The high shear mixer can thoroughly mix the components of a mixture at a high speed, while the low shear mixer mixes the components of a mixture at a low speed.

The first extraction step may be conducted at a temperature of about 12.8° C. (55° F.) to about 68° C. (155° F.), and preferably from about 23° C. (74° F.) to about 35° C. (95° F.). During the course of the present invention, it was determined that if the first extraction step is conducted at relatively high temperatures, i.e. above about 52° C. (125° F.), the lecithin tends to emulsify the oil and water together, which can make separation of the oil from the lecithin difficult.

The first extraction step can be carried out over a pH range of from about 2.5 to about 10.0, and more particularly over a pH range of from about 4.5 to about 7.5. It was determined that a neutral pH was particularly useful for purposes of the present invention, however, acceptable separation of oils from the lecithin can be achieved at a pH of 3.5. At higher pH's, e.g. above about 9.5, the desired separation becomes more problematic.

The residual lecithin material may be recovered from the first (and second) extraction step by centrifuging the mixture. The liquid phase may be separated from the solids and analyzed.

Under specific conditions, approximately 32 wt. % of the starting lecithin may be removed in the form of oil and sugars during the first extraction step.

The second extraction step may be performed under the same conditions with fresh solvent, i.e. the ethanol/water blend. The ratio of the ethanol to water used in second extraction step can generally be from about 3:1 to about 1:3, and preferably from about 2:1 to about 1:1. A ratio of about 5:4 was found to be particularly useful for purposes of the present invention.

The second extraction step results in the net removal of another 3-4 wt. % of the starting lecithin, predominately in the form of sugars.

After the two extraction steps, and when all of the water has been removed, the residual lecithin has an oil and sugar content of about 10 wt. % or less and a yield of approximately 65 wt. %. Only about 1.5 wt. % of the phospholipids are removed as a result of the first and second extraction steps.

The residual material may contain phospholipid content of up to 75 wt. %, which may include a phosphatidyl-choline (PC) content of 24.5 wt. %, a phosphatidyl-ethanolamine (PE) content of 19.6 wt. %, and a phosphatidyl-inositol (PI) content of 14.3 wt. %.

It has been found that if the crude lecithin is mixed with the ethanol/water blend at a temperature of about 27° C. to about 35° C. in a low shear mixer, the separation of sugar is more efficient than the separation of oil. Under this specific condition, only trace amounts of oil are removed, while substantial amounts of sugar can be removed. The resulting lecithin product may contain less than 1.0 wt. % of sugar content. This improved lecithin product retains fluidity and high heat resistance.

The separation of the solid lecithin material from the extract can be performed by any one of a number of physical separation means including a screw-press or expeller, a rolling mill, a plate press, a rotary filter and a centrifuge. The separation of the solid lecithin material from the extract as well as the mixing of the lecithin material with the blend(s) of ethanol and water can be conducted in a batch fashion or a continuous manner, using conventional processing equipment.

The improved lecithin recovered from the second extraction step can be dried at a temperature from about 38° C. (100° F.) to about 104° C. (220° F.), and more particularly at a temperature of from about 52° C. (125° F.) to about 74° C. (165° F.). Any conventional drying apparatus can be utilized to dry the reduced oil and sugar lecithin product, such as, for example, an oven. It is also possible to granulate the resulting reduced oil and sugar lecithin product, adding anti-caking agent(s) thereto as necessary.

These and other aspects of the present invention may be more readily understood by reference to the following example. In the example and throughout, percentages are by weight unless otherwise indicated.

### EXAMPLE 1

In this Example, 200 g lecithin were mixed at a 3:1 ratio with a 5:4 blend of ethanol (333 g) and water (267 g) at about 23° C. (74° F.) in a high shear mixer for 20 minutes. The resulting mixture was centrifuged to remove 624.6 g extract which contained about 300 g ethanol, about 250 g water, 60.4 g sugars, oils and phospholipids (5 wt. %). 175.4 g lecithin (moist with ethanol and water) were obtain after centrifuging and mixed at a 1.5:1 ratio with a 1.6:1.3 blend of ethanol (280 g) and water (175 g). The resulting mixture was centrifuged to remove 285 g extract which contained about 155 g ethanol, about 125 g water, 8.5 g sugars and oils. 169.5 g lecithin (moist with ethanol and water) were obtain after centrifuging. This final reduced oil and sugar lecithin product had a phospholipids content of 75 wt. %, a phosphatidyl-choline (PC) content of 24.5 wt. %, a phosphatidyl-ethanolamine (PE) content of 19.6 wt. %, a phosphatidyl-inositol (PI) content of 14.3 wt. %, and an acetone insolubles content of 89.

### EXAMPLE 2

In this example, 200 g soy bean lecithin were mixed at a 3:1 ratio with a 1:1 blend of ethanol (300 g) and water (300 g) at 23° C. (74° F.), which was adjusted to pH 3.5. This was then mixed with a high shear mixer for 30 minutes. The resulting mixture was centrifuged to remove 587 g extract which contained about 61.14 g sugars, and oils. 182.2 g lecithin (moist with ethanol and water) were obtained after centrifuging and mixed with 273.2 g of a 1.6:1.3 blend of ethanol (150.7 g) and water (122.5 g) for 20 minutes. The resulting mixture was centrifuged to remove 266 g extract which contained about 262.5 g ethanol and water, and 3.2 g sugars and oils. 165.8 g lecithin (moist with ethanol and water) were obtained after centrifuging. The final oil reduced lecithin product had phospholipids content of 55 wt. %, a phosphatidyl-choline (PC) content of 28 wt. %, a phosphatidyl-ethanolamine (PE) content of 23 wt. %, a phosphatidyl-inositol (PI) content of 4 wt. %, and an acetone insolubles content of 84.

### EXAMPLE 3

In this example, 200 g lecithin were mixed at a 2:1 ratio with a 5:4 blend of ethanol (222 g) and water (178 g). This mixture was adjusted to pH 4.0 and heated to 48.9° C. (120° F.) and mixed in a high shear mixer for 20 minutes. The resulting mixture was centrifuged to remove 383 g extract which contained about 185 g ethanol, about 198 g water, 62 g of sugars, and oils. 190.9 g lecithin (moist with ethanol and water) were obtained after centrifuging. This was then blended at a 1.5:1 ratio with a 1.6:1.3 blend of ethanol (280 g) and water (175 g) and again adjusted to 4.0. This blend was mixed for approximately 30 minutes. The resulting mixture was centrifuged to remove 286 g extract which contained about 155 g ethanol, about 125 g water, 8.5 g sugars and oils. 168 g lecithin (moist with ethanol and water) were obtained after centrifuging. This material was blended with ethanol at a 2:1 ratio and again adjusted to pH 4.0, and mixed. This mixture was separated via centrifugation. The extract consisted of 407 g of ethanol, water, and approximately 28 g of lipid material comprising about 14 wt % phosphatidyl-choline (PC), and 16 wt. % phosphatidyl-ethanolamine (PE). The substantially oil and sugar free residual lecithin product had a phospholipids content of about 36 wt. %, a phosphatidyl-choline (PC) content of 9 wt. %, a phosphatidyl-ethanolamine (PE) content of 20 wt. %, a phosphatidyl-inositol (PI) content of 7 wt. %, and an acetone insolubles content of 93.

### EXAMPLE 4

In this example, 200 g lecithin were mixed at a 3:1 ratio with a 5:4 blend of ethanol (333 g) and water (267 g) and the pH was adjusted to 2.5. This was mixed at 23° C. (74° F.) for 45 minutes. The resulting mixture was centrifuged to remove 540.8 g extract which contained about 325 g ethanol, about 215 g water, 29 g sugars and oils. 223.9 g lecithin (moist with ethanol and water) were obtained upon separation. This was then mixed at a 1.5:1 ratio with a 1.6:1.3 blend of ethanol (280 g) and water (175 g), and adjusted to a pH of 4.0. The resulting mixture was separated and 223 g extract was removed which consisted of ethanol with about 45% water, and 6.7 g sugars. 169.5 g lecithin (moist with ethanol and water) were obtained after centrifuging. This final improved lecithin product had a phospholipids content of 70 wt. %, a phosphatidyl-choline (PC) content of 22 wt. %, a phosphatidyl-ethanolamine (PE) content of 21 wt. %, a phosphatidyl-inositol (PI) content of 15 wt. %, and an acetone insolubles content of 88.

### EXAMPLE 5

In this example, 250 g lecithin were mixed at a 1:1 ratio with an ethanol and water blend that had 45% moisture. This was mixed at 35° C. (95° F.) for 60 minutes. The resulting mixture was allowed to separate and the upper phase was decanted off. This extract, consisting of 261 g, contained about 43% water and approximately 8 g sugars with only slight traces of oil, and the balance was ethanol. The heavy phase consisted of about 415 g of lecithin and oil, moist with ethanol and water. This lecithin concentrated material was then again mixed with an ethanol and water blend that had 45% moisture at a 1.6:1 ratio of lecithin to solvent, and adjusted to a pH of 3.0. This was mixed for 20 minutes before the acid was neutralized and the pH brought back to 6.0. The resulting mixture was again allowed to separate and the top layer decanted, with this light phase comprising of 390 g which consisted of ethanol with about 43% water, and 11.7 g sugars. The heavy, or retained layer, wherein all the phosphatides were concentrated, consisted of 620 g lecithin moist with ethanol and water. The ethanol and water were then evaporated off. This final lecithin product had an acetone insolubles content of 72, and a residual sugar content of approximately 0.8%.

### EXAMPLE 6

In this example, 250 g. lecithin were mixed at a 3:1 ratio with an ethanol and water blend that had 45% moisture. This was mixed with a low shear mixer at 27° C. for 30 minutes. The resulting mixture was allowed to separate and the upper phase was decanted off. This extract, consisting of 612 g, contained about 54% water and approximately 22 g sugars and oil, and the balance was ethanol. The heavy phase consisted of about 388 g of lecithin and oil, moist with ethanol and water. This lecithin concentrated material was then again mixed with an ethanol and water blend that had 45% moisture at a 3:1 ratio of lecithin to solvent, and adjusted to a pH of 3.0. This was mixed for 20 minutes before the acid was neutralized and the pH brought back to 6.0. The resulting mixture was again allowed to separate and the top layer decanted, with this light phase comprising of 767 g which consisted of ethanol with about 43% water, and 12.5 g sugars. The heavy, or retained layer, wherein all the phosphatides were concentrated, consisted of 210 g lecithin moist with ethanol and water. The ethanol and water were then evaporated off. This final lecithin product had an acetone insoluble content of 68.5, and a residual sugar content of approximately 0.4%.

While this invention has been described as having exemplary formulations, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A lecithin product, comprising:
(a) phospholipids of up to about 75 wt. % of total dry matter; and
(b) oil and sugar content of about 10 wt. % or less of total dry matter.

2. The lecithin product of claim 1, wherein the weight percent of phospholipids is about 75 wt. % of total dry matter.

3. The lecithin product of claim 1, wherein the weight percent of sugar is less than about 1.0 wt. % of total dry matter.

4. The lecithin product of claim 1, wherein the product is granulated.

5. A lecithin product, comprising:
(a) an acetone insolubles content of more than 68; and
(b) sugar content of less than 1.0 wt. % of total dry matter.

6. A liquid or dry beverage, food or nutritional product that includes the lecithin product of claim 1.

7. A liquid or dry beverage, food or nutritional product that includes the lecithin product of claim 5.

8. A method for producing an improved lecithin product comprising the steps of:
(a) providing a crude lecithin material;
(b) mixing the crude lecithin material with a blend of ethanol and water to form a first mixture;
(c) retaining solids from step (b);
(d) mixing the retained solids in step (c) with a blend of ethanol and water to form a second mixture; and
(e) retaining solids from step (d) as an improved lecithin product.

9. The method according to claim 8, wherein a weight ratio of ethanol to water in the blend of ethanol and water in at least one of steps (b) and (d) is from about 3:1 to about 1:3.

10. The method according to claim 9, wherein the weight ratio of ethanol to water in the blend of ethanol and water in at least one of steps (b) and (d) is about 5:4.

11. The method according to claim 8, wherein a weight ratio of the crude lecithin material to the blend of ethanol and water in step (b) is from about 1:1 to about 1:5.

12. The method according to claim 11, wherein the weight ratio of the crude lecithin material to the blend of ethanol and water in step (b) is about 1:4.

13. The method according to claim 8, wherein a weight ratio of the retained solids to the blend of ethanol and water in step (d) is from about 1:1 to about 1:6.

14. The method according to claim 13, wherein the weight ratio of the retained solids to the blend of ethanol and water in step (d) is about 1:4.

15. The method according to claim 8, wherein steps (b) and (d) are preformed at a temperature of from about 12.8° C. to about 68° C.

16. The method according to claim 15, wherein step (b) and optionally (d) is preformed at a temperature of about 23° C., and in a high shear mixer.

17. The method according to claim 16, wherein the solids are retained in steps (c) and (e) by centrifuging.

18. The method according to claim 17, further comprising the step of drying the improved lecithin product.

19. The method according to claim 18, wherein the improved lecithin product in step (e) comprises phospholipids of up to 75 wt. % of total dry matter, and oil and sugar content of less than about 10 wt. %.

20. The method according to claim 18, further comprising the step of granulating the improved lecithin product.

21. The method according claim 15, wherein step (b) and optional step (d) is preformed at a temperature of about 27° C. to about 35° C., and in a low shear mixer.

22. The method according to claim 21, wherein the solids are retained in steps (c) and (e) by allowing the solids of step (c) and (e) to separate from liquid phase, and decanting the liquid phase.

23. The method according to claim 22, further comprising the step of drying the improved lecithin product.

24. The method according to claim 23, wherein the improved lecithin product comprises an acetone insolubles content of more than 68 and sugar content of less than 1.0 wt. %.

25. The improved lecithin product derived from the method of claim 8.

26. The improved lecithin product derived from the method of claim 19.

27. The improved lecithin product derived from the method of claim 24.
